(19) United States
(12) Patent Application Publication
(10) Pub. No.: US 2024/0342288 A1
(43) Pub. Date: Oct. 17, 2024

(54) METHODS OF ANTIBODY PRODUCTION

(71) Applicant: AMGEN INC., Thousand Oaks, CA (US)

(72) Inventors: Ashutosh Sharma, Thousand Oaks, CA (US); Bryan Dransart, Thousand Oaks, CA (US)

(21) Appl. No.: 18/525,039

(22) Filed: Nov. 30, 2023

Related U.S. Application Data
(63) Continuation of application No. 17/267,425, filed on Feb. 9, 2021, now abandoned.
(60) Provisional application No. 62/717,357, filed on Aug. 10, 2018.

(51) Int. Cl. A61K 47/02; A61K 45/06; A61K 47/08; A61K 47/26

(52) U.S. Cl. CPC A61K 47/02; A61K 45/06; A61K 47/08; A61K 47/26

(57) ABSTRACT

The present disclosure provides materials and methods for preparing an antibody pharmaceutical formulation having a viscosity of 10 cP or less comprising exchanging a composition comprising the antigen binding protein with a diafiltration buffer comprising calcium at a temperature greater than 30° C.

The present application claims the benefit of priority to U.S. Provisional Application No. 62/717,357, filed Aug. 10, 2018 the disclosure of which are incorporated herein by reference in their entireties.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: ASCII (text) file named “53022A_SeqListing.txt,” 17,859 bytes, created on Aug. 8, 2019.

### INCORPORATION BY REFERENCE

The following applications are hereby incorporated by reference in their entirety: International Patent Application No. PCT/US2012/049331, filed Aug. 2, 2012, which claims priority to U.S. Provisional Patent Application No. 61/515,191, filed Aug. 4, 2011; U.S. patent application Ser. No. 11/410,540, filed Apr. 25, 2006, which claims priority to U.S. Provisional Patent Application No. 60/792,645, filed Apr. 17, 2006, U.S. Provisional Patent Application No. 60/782,244, filed Mar. 13, 2006, U.S. Provisional Patent Application No. 60/776,847, filed Feb. 24, 2006, and U.S. Provisional Patent Application No. 60/677,583, filed May 3, 2005; and U.S. patent application Ser. No. 11/411,003 (issued as U.S. Pat. No. 7,592,429), filed Apr. 25, 2006, which claims priority to U.S. Provisional Patent Application No. 60/792,645, filed Apr. 17, 2006, U.S. Provisional Patent Application No. 60/782,244, filed Mar. 13, 2006, U.S. Provisional Patent Application No. 60/776,847, filed Feb. 24, 2006, and U.S. Provisional Patent Application No. 60/677,583, filed May 3, 2005. The following applications also are hereby incorporated by reference: U.S. patent application Ser. No. 12/212,327, filed Sep. 17, 2008, which claims priority to U.S. Provisional Patent Application No. 60/973,024, filed Sep. 17, 2007; and U.S. patent application Ser. No. 12/811,171, filed Jun. 29, 2010, which is a U.S. National Phase Application pursuant to 35 U.S.C. § 371 of International Patent Application No. PCT/US08/86864, filed on Dec. 15, 2008, which claims priority to U.S. Provisional Patent Application No. 61/013,917, filed Dec. 14, 2007.

### BACKGROUND

Highly concentrated liquid antibody formulations are useful for delivering a dose of therapeutic in smaller volume of carrier. However, highly concentrated protein formulations pose several problems, including instability due to the formation of particulates and increased viscosity as a result of numerous intermolecular interactions from the macromolecular nature of antibodies. Highly viscous formulations also are difficult to manufacture, draw into a syringe, and inject. The use of force in manipulating the viscous formulations leads to excessive frothing, which can lead to denaturation and inactivation of active biologics.

### SUMMARY OF THE INVENTION

In one aspect, described herein is a method of preparing an antibody pharmaceutical formulation having a viscosity of 10 cP or less comprising buffer exchanging a composition comprising the antibody with a diafiltration buffer comprising a calcium salt at a temperature greater than 30° C. In some embodiments, the antibody is romosozumab, abciximab, adalimumab, alemtuzumab, basiliximab, belimumab, bevacizumab, brentuximab vedotin, canakinumab, cetuximab, certolizumab pegol, daclizumab, denosumab, eculizumab, efalizumab, gemtuzumab, golimumab, ibritumomab tiuxetan, infliximab, ipilimumab, muromonab-CD3, natalizumab, nivolumab, ofatumumab, omalizumab, palivizumab, panitumumab, ranibizumab, rituximab, tocilizumab, tositumomab, trastuzumab or ustekinumab, vedolizumab.

In some embodiments, the exchanging step occurs via ultrafiltration/diafiltration. In some embodiments, the composition comprises the antibody at a concentration of least 70 mg/mL, at least 71 mg/mL, at least 72 mg/mL, at least 73 mg/mL. at least 74 mg/mL, at least 75 mg/mL, at least 76 mg/mL, at least 77 mg/mL, at least 78 mg/mL, at least 79 mg/mL, at least 80 mg/mL, at least 81 mg/mL, at least 82 mg/mL, at least 83 mg/mL, at least 84 mg/mL, at least 85 mg/mL, at least 86 mg/mL, at least 87 mg/mL, at least 88 mg/mL, at least 89 mg/mL, at least 90 mg/mL, at least 91 mg/mL, at least 92 mg/mL, at least 93 mg/mL, at least 94 mg/mL, at least 95 mg/mL, at least 96 mg/mL, at least 97 mg/mL, at least 98 mg/mL, at least 99 mg/mL, at least 100 mg/mL, at least 101 mg/mL, at least 102 mg/mL, at least 103 mg/mL, at least 104 mg/mL, at least 105 mg/mL, at least 106 mg/mL, at least 107 mg/mL, at least 108 mg/mL, at least 109 mg/mL, at least 110 mg/mL, at least 111 mg/mL, at least 112 mg/mL, at least 113 mg/mL, at least 114 mg/mL, at least 115 mg/mL, at least 116 mg/mL, at least 117 mg/mL, at least 118 mg/mL, at least 119 mg/mL, or at least 120 mg/mL, In some embodiments, the composition comprises the antibody at a concentration ranging from 70 mg/mL to 210 mg/mL. In some embodiments, the composition comprises the antibody at a concentration of less than 210 mg/mL. In some embodiments, the exchanging step occurs at a temperature between 30° C. and 40° C. (e.g., 37° C.), or greater than 35° C.

In some embodiments, the calcium salt is calcium acetate.

In some embodiments, the diafiltration buffer comprises at least 20 mM (e.g., about 23 mM) calcium acetate.

The some embodiments, the diafiltration buffer further comprises a polyol (e.g., sucrose), optionally at a concentration of about 1% to about 15%. In some embodiments, the diafiltration buffer comprises sucrose at a concentration of about 7%.

In some embodiments, the antibody pharmaceutical formulation after the exchanging step comprises about 50 mM acetate and about 12 mM calcium.

The methods of the disclosure optionally further comprise the step of filtering and/or aliquoting the antibody pharmaceutical formulation into a drug product form.

The terms “comprising,” “having,” “including,” and “containing” are to be construed as open-ended terms (i.e., meaning “including, but not limited to,” and permit the presence of one or more features or components) unless otherwise noted. It should be understood that while various embodiments in the specification are presented using “comprising” language, under various circumstances, a related embodiment may also be described using “consisting of” or “consisting essentially of” language. It is to be noted that the term “a” or “an” refers to one or more, for example, “an immunoglobulin molecule,” is understood to represent one or more immunoglobulin molecules, unless context dictates otherwise. As such, the terms “a” (or “an”), “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It should also be understood that when describing a range of values, the characteristic being described could be an individual value found within the range. For example, “a pH from about pH 4 to about pH 6,” could be, but is not limited to, pH 4, 4.2, 4.6, 5.1, 5.5, etc. and any value in between such values. Additionally, “a pH from about pH 4 to about pH 6,” should not be construed to mean that the pH of a formulation in question varies 2 pH units in the range from pH 4 to pH 6 during storage, but rather a value may be picked in that range for the pH of the solution, and the pH remains buffered at about that pH.

In any of the ranges described herein, the endpoints of the range are included in the range. However, the description also contemplates the same ranges in which the lower and/or the higher endpoint is excluded. Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole.

All references cited herein are hereby incorporated by reference in their entireties.

### BRIEF DESCRIPTION OF THE FIGURES

FIG. 1 is a graph showing the effect of calcium acetate at various concentrations on the viscosity of an antibody composition. Viscosity (cP. Y-axis) is plotted against concentration of antibody (mg/mL, X-axis).

FIG. 2 provides the ultrafiltration parameters in the absence of calcium acetate.

FIG. 3 provides the ultrafiltration parameters in the presence of calcium acetate.

FIG. 4 is a graph showing the effect of temperature on the viscosity of an antibody composition. Feed pressure (psi, Y-axis) is plotted against retentate concentration (mg/mL, X-axis).

### DETAILED DESCRIPTION

The present disclosure is based on the discovery that buffer exchanging a composition comprising an antibody with a diafiltration buffer comprising calcium salt at a temperature greater than 30° C. results in an antibody pharmaceutical formulation having a viscosity of 10 cP or less.

The ability to formulate antibodies at a higher concentration provides improved patient dosing regimes, smaller injection volumes, a wider range of device options, and improvements in supply chain considerations. Many antibodies are very viscous at higher concentrations can limit processing and drug delivery options. In particular, antibody compositions with high viscosity can be problematic for a final drug substance ultrafiltration/diafiltration process step due to long processing times, high pressures, large membrane areas required for processing, and potentially poor product recoveries. As described herein, the combination of increased temperature and calcium salt (e.g., calcium acetate) is surprisingly effective at reducing the viscosity of the composition considering that the solubility of calcium salt decreases with increasing temperature.

As used herein, the terms “ultrafiltration” or “UF” refers to any technique in which a solution or a suspension is subjected to a membrane (e.g., semi-permeable membrane) for separating a product (e.g., a protein) from other materials in a solution or suspension. An ultrafiltration membrane, for example, retains molecules that are larger than the pores of the membrane while smaller molecules such as salts, solvents and water freely pass through the membrane. The solution retained by the membrane is referred to as a “concentrate” or “retentate,” while the solution that passes through the membrane is referred to as a “filtrate” or “permeate.” Ultrafiltration may be used to increase the concentration of macromolecules in a solution or suspension. In an aspect, ultrafiltration is used to increase the concentration of a protein in a solution.

Membrane filters, such as ultrafiltration membranes, of the present disclosure may have a pore size of 0.001 to 0.1 micron. In some embodiments, a membrane filter has a pore size of 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095 or 0.100 micron. In some embodiments, membrane filters of the present disclosure have a molecular cutoff value of 15 kilodaltons (kDa) to 50 kDa, or more. For example, in some embodiments, a membrane filter has a molecular cut-off value of 15 kDa, 20 kDa, 25 kDa, 30 kDa, 35 kDa, 40 kDa, 45 kDa or 50 kDa, or any intermediate value. In some embodiments, the molecular weight cut off of the membrane is 30 kDa.

As used herein, the term “diafiltration” or “DF” is used to refer to, for example, using an ultrafiltration membrane to remove, replace, or lower the concentration of salts or solvents from solutions or mixtures containing proteins, peptides, nucleic acids, or other biomolecules. Diafiltration may or may not lead to an increase in the concentration of retained components, including, proteins. For example, in continuous diafiltration, a solvent is continuously added to the retentate at the same rate as the filtrate is generated. In this case, the retentate volume and the concentration of retained components does not change during the process. On the other hand, in discontinuous or sequential dilution diafiltration, an ultrafiltration step is followed by the addition of solvent to the retentate side; if the volume of solvent added to the retentate side is not equal or greater to the volume of filtrate generated, then the retained components will have a high concentration. Diafiltration may be used to alter the pH, ionic strength, salt composition, buffer composition, or other properties of a solution or suspension of macromolecules.

As used herein, the terms “ultrafiltration/diafiltration/” or “UF/DF” refer to any process, technique or combination of techniques that accomplishes ultrafiltration and/or diafiltration, either sequentially or simultaneously.

In some embodiments, the viscosity of the composition comprising the antibody is measured prior to the buffer exchange step, and viscosity of the resulting formulation is measured after buffer exchanging the starting composition with the diafiltration buffer comprising a calcium salt at a temperature greater than 30° C. Methods of measuring viscosity are well known in the art and include, for example, use of a capillary viscometer or a cone-plate rheometer. Any methods may be used provided the same method is used to compare the starting composition and the resulting formulation.

The term “viscosity” as used herein refers to “absolute viscosity.” Absolute viscosity, sometimes called dynamic or simple viscosity, is the product of kinematic viscosity and fluid density: Absolute Viscosity=Kinematic Viscosity×Density. The dimension of kinematic viscosity is L²/T where L is a length and T is a time. Commonly, kinematic viscosity is expressed in centistokes (cSt). The SI unit of kinematic viscosity is mm²/s, which is 1 cSt. Absolute viscosity is expressed in units of centipoise (cP). The SI unit of absolute viscosity is the millipascal-second (mPa-s), where 1 cP=1 mPa-s.

Viscosity measurements may be made at a storage or administration temperature, e.g. 2-8° C. or 25° C. (room temperature). In some embodiments, absolute viscosity of the resulting pharmaceutical composition at the storage and/or administration temperature is 15 cP or less, 14 cP or less, 13 cP or less, 12 cP or less, 11 cP or less, 10 cP or less, 9 cP or less, 8 cP or less, 7 cP or less, 6 cP or less, 5 cP or less, or 4 cP or less.

A “diafiltration buffer” is a buffer that does not itself contain the antibody but is used to make a formulation comprising the antibody. The diafiltration buffer comprises a calcium salt. Exemplary calcium salts include, but are not limited to, calcium acetate, calcium carbonate, calcium citrate, calcium gluconate, calcium lactate, calcium glutamate, calcium succinate, and calcium chloride. In some embodiments, the calcium salt is present in the diafiltration buffer at a concentration ranging from 5 mM to 150 mM. In some embodiments, the calcium salt is present in the diafiltration buffer at a concentration ranging from 10 mM to 30 mM. In some embodiments, the calcium salt is present in the diafiltration buffer at a concentration of at least 10 mM, at least 11 mM, at least 12 mM, at least 13 mM, at least 14 mM, at least 15 mM, at least 16 mM, at least 17 mM, at least 18 mM, at least 19 mM, at least 20 mM, at least 21 mM, at least 22 mM, at least 23 mM, at least 24 mM, at least 25 mM, at least 26 mM, at least 27 mM, at least 28 mM, at least 29 mM or at least 30 mM. In certain embodiments, the concentration of calcium salt in the diafiltration buffer is not greater than about 20 mM, not greater than about 21 mM, not greater than about 22 mM, not greater than about 23 mM, not greater than about 24 mM, not greater than about 25 mM, not greater than about 26 mM, not greater than about 27 mM, not greater than about 28 mM, not greater than about 29 mM or not greater than about 30 mM. Any range featuring a combination of the foregoing endpoints is contemplated, including but not limited to, from about 0.5 mM to about 30 mM, from about 20 mM to about 30 mM, or from about 20 mM to about 25 mM. In some embodiments, the calcium salt is present in the diafiltration buffer at a concentration that reduces viscosity of an antibody composition resulting from the buffer exchange step disclosed herein by at least 10%, at least 20%, at least 30%, at least 40%, at least 50%, at least 60%, at least 70%, at least 80% or more compared to the antibody composition prior to the buffer exchange with the diafiltration buffer comprising calcium salt at a temperature greater than 30° C., or that achieves a viscosity of 10 cP or less, 9 cP or less, 8 cP or less, 7 cP or less, 6 cP or less, or 5 cP or less.

In all of the ranges described herein, the concentration of cation, anion or salt described herein is relevant to the diafiltration buffer. In any of the ranges described herein, the endpoints of the range are included in the range. However, the description also contemplates the same ranges in which the lower and/or the higher endpoint is excluded.

In some embodiments, the diafiltration buffer described herein further comprises, in addition to the calcium salt, a buffer (e.g., an acetate buffer) at a concentration of at least about 5 mM, at least about 6 mM, at least about 7 mM, at least about 8 mM, at least about 9 mM, at least about 10 mM, or at least about 15 mM. In some embodiments, the concentration is no greater than about 10 mM, no greater than about 15 mM, no greater than about 20 mM, no greater than about 25 mM, no greater than about 30 mM, no greater than about 35 mM, no greater than about 40 mM, no greater than about 45 mM or no greater than about 50 mM. Any range featuring a combination of the foregoing endpoints is contemplated, including but not limited to from about 5 mM to about 15 mM, or from about 5 mM to about 10 mM, or from about 20 mM to about 30 mM, or from about 20 mM to about 25 mM. The buffer is preferably added to a concentration that maintains pH around 5-6 or 5-5.5 or 4.5-5.5. When the calcium salt in the formulation is calcium acetate, in some embodiments, the total concentration of acetate is about 10 mM to about 60 mM, or about 20 mM to about 40 mM.

In some aspects, the diafiltration buffer comprises a total concentration of acetate that is at least about 10 mM, at least about 15 mM, at least about 20 mM, at least about 25 mM, at least about 30 mM, at least about 35 mM, at least about 40 mM, at least about 45 mM, or at least about 50 mM. In some embodiments, the concentration of acetate is no greater than about 30 mM, no greater than about 35 mM, no greater than about 40 mM, no greater than about 45 mM, no greater than about 50 mM, no greater than about 55 mM, no greater than about 60 mM, no greater than about 65 mM, no greater than about 70 mM, no greater than about 75 mM, no greater than about 80 mM, no greater than about 85 mM, or no greater than about 90 mM. Any range featuring a combination of the foregoing endpoints is contemplated, including but not limited to: about 10 mM to about 50 mM, about 20 mM to about 50 mM, about 20 mM to about 40 mM, about 30 mM to about 50 mM, or about 30 mM to about 75 mM. By way of nonlimiting example, a solution containing 10 mM calcium acetate will have 20 mM acetate anion and 10 mM of calcium cation, because of the divalent nature of the calcium cation, while a solution containing 10 mM sodium acetate will have 10 mM sodium cation and 10 mM acetate anion.

In some embodiments, the diafiltration buffer comprises a glutamate buffer or a succinate buffer at a concentration of at least about 5 mM, at least about 6 mM, at least about 7 mM, at least about 8 mM, at least about 9 mM, at least about 10 mM, or at least about 15 mM. In some embodiments, the concentration is no greater than about 10 mM, no greater than about 15 mM, no greater than about 20 mM, no greater than about 25 mM, no greater than about 30 mM, no greater than about 35 mM, no greater than about 40 mM, no greater than about 45 mM or no greater than about 50 mM. Any range featuring a combination of the foregoing endpoints is contemplated, including but not limited to from about 5 mM to about 15 mM, or from about 5 mM to about 10 mM, or from about 20 mM to about 30 mM, or from about 20 mM to about 25 mM. The buffer is preferably added to a concentration that maintains pH around 5-6 or 5-5.5 or 4.5-5.5.

In some embodiments, the total concentration of ions (cations and anions) in diafiltration buffer is at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, or at least about 85 mM. In some embodiments, the total concentration of ions is no greater than about 30 mM, no greater than about 35 mM, no greater than about 40 mM, no greater than about 45 mM, no greater than about 50 mM, no greater than about 55 mM, no greater than about 60 mM, no greater than about 65 mM, no greater than about 70 mM, no greater than about 75 mM, no greater than about 80 mM, no greater than about 85 mM, no greater than about 90 mM, no greater than about 95 mM, no greater than about 100 mM, no greater than about 110 mM, no greater than about 120 mM, no greater than about 130 mM, no greater than about 140 mM, no greater than about 150 mM, no greater than about 160 mM, no greater than about 170 mM, no greater than about 180 mM, no greater than about 190 mM or no greater than about 200 mM. Any range featuring a combination of the foregoing endpoints is contemplated, including but not limited to: about 30 mM to about 60 mM, or about 30 mM to about 70 mM, or about 30 mM to about 80 mM, or about 40 mM to about 150 mM, or about 50 mM to about 150 mM. By way of nonlimiting example, a solution of 10 mM calcium acetate will have a 30 mM total concentration of ions (10 mM cations and 20 mM anions).

In various embodiments, the antibody composition is buffer exchanged with the diafiltration buffer at a temperature greater than 30° C. In some embodiments, the buffer exchange occurs at a temperature between 30° C. and 40° C. In some embodiments, the buffer exchange occurs at a temperature greater than 35° C. In some embodiments, the buffer exchange occurs at 30° C., 31° C., 32° C., 33° C., 34° C., 35° C., 36° C., 37° C., 38° C., 39° C. or 40° C. In some embodiments, the buffer exchange occurs at 37° C.

The diafiltration buffer described herein optionally comprises at least one polyol. Polyols encompass a class of excipients that includes sugars (e.g., mannitol, sucrose, or sorbitol) and other polyhydric alcohols (e.g., glycerol and propylene glycol). The polymer polyethylene glycol (PEG) is included in this category. Polyols are commonly used as stabilizing excipients and/or isotonicity agents in both liquid and lyophilized parenteral protein formulations. Polyols can protect proteins from both physical and chemical degradation pathways.

Exemplary polyols include, but are not limited to, sucrose, trehalose, mannose, maltose, lactose, glucose, raffinose, cellobiose, gentiobiose, isomaltose, arabinose, glucosamine, fructose, mannitol, sorbitol, glycine, arginine HCL, poly-hydroxy compounds (including, e.g., polysaccharides such as dextran, starch, hydroxyethyl starch, cyclodextrins, captisol, N-methyl pyrollidene, cellulose and hyaluronic acid), and sodium chloride (Carpenter et al., Develop. Biol. Standard 74:225, (1991)).

Additional polyols include, but are not limited to, propylene glycol, glycerin (glycerol), threose, threitol, erythrose, erythritol, ribose, arabinose, arabitol, lyxose, maltitol, sorbitol, sorbose, glucose, mannose, mannitol, levulose, dextrose, maltose, trehalose, fructose, xylitol, inositol, galactose, xylose, fructose, sucrose, 1,2,6-hexanetriol and the like. Higher order sugars include dextran, propylene glycol, or polyethylene glycol. Reducing sugars such as fructose, maltose or galactose oxidize more readily than do non-reducing sugars. Additional examples of sugar alcohols are glucitol, maltitol, lactitol or iso-maltulose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Monoglycosides include compounds obtained by reduction of disaccharides such as lactose, maltose, lactulose and maltulose.

In some embodiments, the at least one polyol is selected from the group consisting of a monosaccharide, a disaccharide, a cyclic polysaccharide, a sugar alcohol, a linear branched dextran, and a linear non-branched dextran, or combinations thereof. In some embodiments, the at least one polyol is a disaccharide selected from the group consisting of sucrose, trehalose, mannitol, and sorbitol or a combination thereof.

In some embodiments, the diafiltration buffer comprises at least one polyol (e.g., saccharide) at a concentration of about 0% to about 40% w/v, or about 0% to about 20% w/v, or about 1% to about 15% w/v. In some embodiments, the diafiltration buffer comprises at least one polyol (e.g., saccharide) at a concentration of at least 0.5, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, or at least 40% w/v. In some embodiments, the diafiltration buffer comprises at least one polyol (e.g., saccharide) at a concentration of about 1, about 2, about 3, about 4, about 5, about 6, about 7, about 8, about 9, about 10, about 11, about 12, about 13, about 14% to about 15% w/v. In some embodiments, the diafiltration buffer comprises at least one polyol (e.g., saccharide) at a concentration of about 1% to about 15% w/v. In a yet further embodiment, the diafiltration buffer comprises at least one polyol (e.g., saccharide) at a concentration of about 9%, about 9.5%, about 10%, about 10.5%, about 11%, about 11.5%, or about 12% w/v. In some embodiments, the diafiltration buffer comprises at least one polyol (e.g., saccharide) at a concentration of about 9% to about 12% w/v. In some embodiments, the at least one polyol (e.g., saccharide) is in the diafiltration buffer at a concentration of about 9% w/v. In some embodiments, the at least one polyol is selected from the group consisting of sucrose, trehalose, mannitol and sorbitol or a combination thereof. In some embodiments, the polyol is sucrose and is present in the diafiltration buffer at a concentration ranging from about 5% to about 9% w/v.

In some embodiments, the diafiltration buffer comprises 20 mM calcium acetate, 7% sucrose. In some embodiments, the pH of the diafiltration buffer ranges from 4-6. In the some embodiments, the pH of the diafiltration buffer is 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9 or 6. In some embodiments, the pH of the diafiltration buffer is 5.1.

### Ultrafiltration/Diafiltration

Ultrafiltration/Diafiltration (also generally referred to herein as UF/DF) selectively utilizes permeable (porous) membrane filters to separate the components of solutions and suspensions based on their molecular size. A membrane retains molecules that are larger than the pores of the membrane while smaller molecules such as salts, solvents and water, which are permeable, freely pass through the membrane. The solution retained by the membrane is known as the concentrate or retentate. The solution that passes through the membrane is known as the filtrate or permeate. One parameter for selecting a membrane for concentration is its retention characteristics for the sample to be concentrated. As a general rule, the molecular weight cut-off (MWCO) of the membrane should be ⅓rd to ⅙th the molecular weight of the molecule to be retained. This is to assure complete retention. The closer the MWCO is to that of the sample, the greater the risk for some small product loss during concentration. Examples of membranes that can be used with methods of the present disclosure include Omega™ PES membrane (30 kDa MWCO, i.e. molecules larger than 30 kDa are retained by the membrane and molecules less than 30 kDa are allowed to pass to the filtrate side of the membrane) (Pall Corp., Port Washington, N.Y.); Pelicon™ 30 kD Regenerated Cellulose Membrane (Millipore Sigma); Millex®.-GV Syringe Driven Filter Unit, PVDF 0.22 μm (Millipore Corp., Billerica, Mass.); Millex®.-GP Syringe Driven Filter Unit, PES 0.22 .mu.m; Sterivex® 0.22 m Filter Unit (Millipore Corp., Billerica, Mass.); and Vivaspin concentrators (MWCO 10 kDa, PES; MWCO 3 kDa, PES) (Sartorius Corp., Edgewood, N.Y.)

There are two forms of UF/DF, including UF/DF in discontinuous mode and UF/DF in continuous mode. The methods of the present disclosure may be performed according to either mode.

Continuous UF/DF (also referred to as constant volume UF/DF) involves washing out the original buffer salts (or other low molecular weight species) in the retentate (sample) by adding water or a new buffer to the retentate at the same rate as filtrate is being generated. As a result, the retentate volume and product concentration does not change during the UF/DF process. The amount of salt removed is related to the filtrate volume generated, relative to the retentate volume. The filtrate volume generated is usually referred to in terms of “diafiltration volumes”. A single diafiltration volume (DV) is the volume of retentate when diafiltration is started. For continuous diafiltration, liquid is added at the same rate as filtrate is generated. When the volume of filtrate collected equals the starting retentate volume, 1 DV has been processed.

Discontinuous UF/DF includes two different methods, discontinuous sequential UF/DF and volume reduction discontinuous UF/DF. Discontinuous UF/DF by sequential dilution involves first diluting the sample with water to a predetermined volume. The diluted sample is then concentrated back to its original volume by UF. Discontinuous UF/DF by volume reduction involves first concentrating the sample to a predetermined volume, then diluting the sample back to its original volume with water or replacement buffer. As with continuous UF/DF, the process is repeated until the level of unwanted solutes, e.g., ionic excipients, are removed.

UF/DF may be performed in accordance with conventional techniques known in the art using water, e.g., WFI, as the UF/DF medium (e.g., Industrial Ultrafiltration Design and Application of Diafiltration Processes, Beaton & Klinkowski, J. Separ. Proc. Technol., 4(2) 1-10 (1983)). Examples of commercially available equipment for performing UF/DF include Millipore Labscale™ TFF System (Millipore), LV Centramate™. Lab Tangential Flow System (Pall Corporation), the UniFlux System (GE Healthcare), FlexAct® UD (Sartorius Stedim Biotech), Mobius® FlexReady TFF (EMD Millipore), Akta™ Readyflux (GE Healthcare), Allegro™ Single-use TFF (Pall Corporation) and stainless steel skids such as Cogent TFF Systems.

The buffer exchanging step with the diafiltration buffer may be performed any number of times, depending on the protein in solution, wherein one diafiltration step equals one total volume exchange. In one embodiment, the diafiltration process is performed 1, 2, 3, 4, 5, 6, 7, 8, 9, 10, 11, 12, 13, 14, 15 or up to as many times are deemed necessary to achieve the desired result. A single round or step of diafiltration is achieved when a volume of diafiltration buffer has been added to the retentate side that is equal to the starting volume of the antibody composition.

In various embodiments, the resulting diafiltered formulation after the exchanging step comprises about 50 mM acetate and about 12 mM calcium.

The methods of the present disclosure also provides a means of concentrating an antigen binding protein at high levels without increasing the viscosity of the resulting diafiltered formulation. The concentration of the antigen binding protein in the aqueous formulation obtained using the methods of the present disclosure can be any amount in accordance with the desired concentration. For example, the concentration of the antigen binding protein in the composition made according to the methods described herein is at least about 70 mg/ml, at least about 71 mg/ml, at least about 72 mg/ml, at least about 73 mg/ml, at least about 74 mg/ml, at least about 75 mg/ml, at least about 76 mg/ml, at least about 77 mg/ml, at least about 78 mg/ml, at least about 79 mg/ml, at least about 80 mg/ml, at least about 81 mg/ml, at least about 82 mg/ml, at least about 83 mg/ml, at least about 84 mg/ml, at least about 85 mg/ml, at least about 86 mg/ml, at least about 87 mg/ml, at least about 88 mg/ml, at least about 89 mg/ml, at least about 90 mg/ml, at least about 91 mg/ml, at least about 92 mg/ml, at least about 93 mg/ml, at least about 94 mg/ml, at least about 95 mg/ml, at least about 96 mg/ml, at least about 97 mg/ml, at least about 98 mg/ml, at least about 99 mg/ml, at least about 100 mg/ml, at least about 101 mg/ml, at least about 102 mg/ml, at least about 103 mg/ml, at least about 104 mg/ml, at least about 105 mg/ml, at least about 106 mg/ml, at least about 107 mg/ml, at least about 108 mg/ml, at least about 109 mg/ml, at least about 110 mg/ml, at least about 111 mg/ml, at least about 112 mg/ml, at least about 113 mg/ml, at least about 114 mg/ml, at least about 115 mg/ml, at least about 116 mg/ml, at least about 117 mg/ml, at least about 118 mg/ml, at least about 119 mg/ml, at least about 120 mg/ml, at least about 121 mg/ml, at least about 122 mg/ml, at least about 123 mg/ml, at least about 124 mg/ml, at least about 125 mg/ml, at least about 126 mg/ml, at least about 127 mg/ml, at least about 128 mg/ml, at least about 129 mg/ml, at least about 130 mg/ml, at least about 131 mg/ml, at least about 132 mg/ml, at least about 132 mg/ml, at least about 133 mg/ml, at least about 134 mg/ml, at least about 135 mg/ml, at least about 136 mg/ml, at least about 137 mg/ml, at least about 138 mg/ml, at least about 139 mg/ml, at least about 140 mg/ml, at least about 141 mg/ml, at least about 142 mg/ml, at least about 143 mg/ml, at least about 144 mg/ml, at least about 145 mg/ml, at least about 146 mg/ml, at least about 147 mg/ml, at least about 148 mg/ml, at least about 149 mg/ml, at least about 150 mg/ml, at least about 151 mg/ml, at least about 152 mg/ml, at least about 153 mg/ml, at least about 154 mg/ml, at least about 155 mg/ml, at least about 156 mg/ml, at least about 157 mg/ml, at least about 158 mg/ml, at least about 159 mg/ml, or at least about 160 mg/ml, and may range up to, e.g., about 300 mg/ml, about 290 mg/ml, about 280 mg/ml, about 270 mg/ml, about 260 mg/ml, about 250 mg/ml, about 240 mg/ml, about 230 mg/ml, about 220 mg/ml, about 210 mg/ml, about 200 mg/ml, about 190 mg/ml, about 180 mg/ml, or about 170 mg/ml. Any range featuring a combination of the foregoing endpoints is contemplated, including but not limited to: about 70 mg/ml to about 250 mg/ml, about 70 mg/ml to about 200 mg/ml, about 70 mg/mL to about 210 mg/mL, about 70 mg/ml to about 160 mg/ml, about 100 mg/ml to about 250 mg/ml, about 100 mg/l to about 200 mg/ml, or about 100 mg/ml to about 180 mg/ml.

An “antigen binding protein” as used herein means a protein that specifically binds a specified antigen. Examples of antigen binding proteins include but are not limited to antibodies, peptibodies, antibody fragments, antibody constructs, fusion proteins, and antigen receptors including chimeric antigen receptors (CARs). The term encompasses intact antibodies that comprise at least two full-length heavy chains and two full-length light chains, as well as derivatives, variants, fragments, and mutations thereof. An antigen binding protein also includes domain antibodies such as nanobodies and scFvs as described further below.

In some embodiments, the antigen binding protein is an antibody. As used herein, the term “antibody” refers to a protein having a conventional immunoglobulin format, comprising heavy and light chains, and comprising variable and constant regions. An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4th ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the invention include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4. IgG1 antibodies are particularly susceptible to reduction of di-sulfide bonds and, as a result, represent one preferred embodiment of the disclosure.

The antibody may be a monoclonal antibody or a polyclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody may be considered as a mammalian antibody, e.g., a mouse antibody, rabbit antibody, goat antibody, horse antibody, chicken antibody, hamster antibody, human antibody, and the like. In certain aspects, the monoclonal antibody is a human antibody. In certain aspects, the monoclonal antibody is a chimeric antibody or a humanized antibody. The term “chimeric antibody” is used herein to refer to an antibody containing constant domains from one species and the variable domains from a second, or more generally, containing stretches of amino acid sequence from at least two species. The term “humanized” when used in relation to antibodies refers to antibodies having at least CDR regions from a non-human source which are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence.

The method of the disclosure also is suitable for obtaining a formulation comprising antigen binding proteins, e.g., antibody fragments such as scFvs, Fabs and VHH/VH, which retain full antigen-binding capacity. Both scFv and Fab are widely used fragments that can be easily produced in prokaryotic hosts. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

In some embodiments, the antigen binding protein is an scFv, Fab VHH/VH, Fv fragment, ds-scFv, scFab, dimeric antibody, multimeric antibody (e.g., a diabody, triabody, tetrabody), miniAb, peptibody VHH/VH of camelid heavy chain antibody, sdAb, a bispecific or trispecific antibody, BsIgG, appended IgG, BsAb fragment, bispecific fusion protein, or BsAb conjugate.

The antigen binding protein may be in monomeric form, or polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

Advantageously, the methods are not limited to the antigen-specificity of the antibody. Accordingly, the antibody (or antibody fragment or antibody protein product) has any binding specificity for virtually any antigen. In exemplary aspects, the antibody binds to a hormone, growth factor, cytokine, a cell-surface receptor, or any ligand thereof. In some embodiments, the antibody is romosozumab, abciximab, adalimumab, alemtuzumab, basiliximab, belimumab, bevacizumab, brentuximab vedotin, canakinumab, cetuximab, certolizumab pegol, daclizumab, denosumab, eculizumab, efalizumab, gemtuzumab, golimumab, ibritumomab tiuxetan, infliximab, ipilimumab, muromonab-CD3, natalizumab, nivolumab, ofatumumab, omalizumab, palivizumab, panitumumab, ranibizumab, rituximab, tocilizumab, tositumomab, trastuzumab, ustekinumab, vedolizumab, or a biosimilar of any of the foregoing.

In some embodiments, the antibody is selected from the group consisting of Muromonab-CD3 (product marketed with the brand name Orthoclone Okt3®), Abciximab (product marketed with the brand name Reopro®.), Rituximab (product marketed with the brand name MabThera®, Rituxan®) (U.S. Pat. No. 5,843,439), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-I131 (product marketed with the brand name Bexxar®), Efalizumab (product marketed with the brand name Raptiva®), Cetuximab (product marketed with the brand name Erbitux®), I'Ibritumomab tiuxetan (product marketed with the brand name Zevalin®), I'Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), I'Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), and Pertuzumab (product marketed with the brand name Perjeta®).

In some embodiments, the antibody is an anti-sclerostin antibody. An “anti-sclerostin antibody” or an “antibody that binds to sclerostin” is an antibody that binds to sclerostin of SEQ ID NO: 1 or portions thereof. Recombinant human sclerostin/SOST is commercially available from, e.g., R&D Systems (Minneapolis, Minn., USA; 2006 Catalog #1406-ST-025). U.S. Pat. Nos. 6,395,511 and 6,803,453, and U.S. Patent Publication Nos. 2004/0009535 and 2005/0106683 (hereby incorporated by reference) refer to anti-sclerostin antibodies generally. Examples of sclerostin antibodies suitable for use in the context of the disclosure also are described in U.S. Patent Publication Nos. 2007/0110747 and 2007/0072797, which are hereby incorporated by reference. Additional information regarding materials and methods for generating sclerostin antibodies can be found in U.S. Patent Publication No. 20040158045 (hereby incorporated by reference).

“Specifically binds” as used herein means that the antibody preferentially binds the antigen over other proteins. In some embodiments, “specifically binds” means the antibody has a higher affinity for the antigen than for other proteins.

In some or any embodiments, the antibody binds to sclerostin of SEQ ID NO: 1, or a naturally occurring variant thereof, with an affinity (Kd) of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M, or less than or equal to 1×10⁻¹² M. Affinity is determined using a variety of techniques, an example of which is an affinity ELISA assay. In various embodiments, affinity is determined by a BIAcore assay. In various embodiments, affinity is determined by a kinetic method. In various embodiments, affinity is determined by an equilibrium/solution method. U.S. Patent Publication No. 2007/0110747 (the disclosure of which is incorporated herein by reference) contains additional description of affinity assays suitable for determining the affinity (Kd) of an antibody for sclerostin.

In various aspects, the antibody comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 2, CDR-H2 has the sequence given in SEQ ID NO: 3, CDR-H3 has the sequence given in SEQ ID NO: 4, CDR-L1 has the sequence given in SEQ ID NO: 5, CDR-L2 has the sequence given in SEQ ID NO: 6 and CDR-L3 has the sequence given in SEQ ID NO: 7. The anti-sclerostin antibody, in various aspects, comprises two of the CDRs or six of the CDRs.

In a preferred embodiment, the anti-sclerostin antibody comprise a set of six CDRs as follows: CDR-H1 of SEQ ID NO: 2, CDR-H2 of SEQ ID NO: 3, CDR-H3 of SEQ ID NO: 4, CDR-L1 of SEQ ID NO: 5, CDR-L2 of SEQ ID NO: 6 and CDR-L3 of SEQ ID NO: 7.

In some or any embodiments, the antibody comprises a light chain variable region comprising an amino acid sequence having at least 75% identity (e.g., at least 75%, 80%, 85%, 90%, 95% or 100% identity) to the amino acid sequence set forth in SEQ ID NO: 8 and a heavy chain variable region comprising an amino acid sequence having at least 75% identity (e.g., at least 75%, 80%, 85%, 90%, 95% or 100% identity) to the amino acid sequence set forth in SEQ ID NO: 9. In various aspects, the difference in the sequence compared to SEQ ID NO: 8 or 9 lies outside the CDR region in the corresponding sequences. In some or any embodiments, the antibody comprises a light chain variable region comprising an amino acid sequence set forth in SEQ ID NO: 8 and a heavy chain variable region comprising an amino acid sequence set forth in SEQ ID NO: 9.

In some or any embodiments, the anti-sclerostin antibody comprises all or part of a heavy chain (e.g., two heavy chains) comprising an amino acid sequence having at least 75% identity (e.g., at least 75%, 80%, 85%, 90%, 95% or 100% identity) to the amino acid sequence set forth in SEQ ID NO: 11 and all or part of a light chain (e.g., two light chains) comprising an amino acid sequence having at least 75% identity (e.g., at least 75%, 80%, 85%, 90%, 95% or 100% identity) to the amino acid sequence set forth in SEQ ID NO 10.

In some or any embodiments, the anti-sclerostin antibody comprises all or part of a heavy chain (e.g., two heavy chains) comprising an amino acid sequence having at least 75% identity (e.g., at least 75%, 80%, 85%, 90%, 95% or 100% identity) to the amino acid sequence set forth in SEQ ID NO: 13 and all or part of a light chain (e.g., two light chains) comprising an amino acid sequence having at least 75% identity (e.g., at least 75%, 80%, 85%, 90%, 95% or 100% identity) to the amino acid sequence set forth in SEQ ID NO 12.

Examples of other anti-sclerostin antibodies include, but are not limited to, the anti-sclerostin antibodies disclosed in International Patent Publication Nos. WO 2008/092894, WO 2008/115732, WO 2009/056634, WO 2009/047356, WO 2010/100200, WO 2010/100179, WO 2010/115932, and WO 2010/130830 (each of which is incorporated by reference herein in its entirety).

It will be understood by one skilled in the art that some proteins, such as antibodies, may undergo a variety of posttranslational modifications. The type and extent of these modifications often depends on the host cell line used to express the protein as well as the culture conditions. Such modifications may include variations in glycosylation, methionine oxidation, diketopiperizine formation, aspartate isomerization and asparagine deamidation. A frequent modification is the loss of a carboxy-terminal basic residue (such as lysine or arginine) due to the action of carboxypeptidases (as described in Harris, RJ. Journal of Chromatography 705:129-134, 1995).

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983], entirely incorporated by reference), acetylation of the N-terminal amine, and amidation of any C-terminal carboxyl group.

A pharmaceutical composition comprising one or more antibodies described herein may be placed within containers (e.g., vials or syringes), along with packaging material that provides instructions regarding the use of such pharmaceutical compositions. Generally, such instructions will include a tangible expression describing the antibody concentration, as well as within certain embodiments, relative amounts of excipient ingredients or diluents (e.g., water, saline or PBS) that may be necessary to reconstitute the pharmaceutical composition.

### Example

This Example describes a representative antibody purification process that uses an elevated temperature buffer exchange (e.g., via ultrafiltration and diafiltration (UF/DF)) step to concentrate and buffer exchange an antibody into a 20 mM Calcium Acetate, 7% Sucrose pH 5.1 diafiltration buffer to produce a final pharmaceutical composition comprising antibody at a concentration of 120 g/L. A surprising outcome for this method is the ability to recover a high concentration protein from overconcentration using both higher temperature (e.g., 37° C.) and calcium salt (e.g., calcium acetate). Calcium acetate has the property of decreasing solubility with increasing temperature (see e.g., Apelblat, A. and Manzurola, E.; J. Chem. Thermodynamics, 1999, 31, 1347-1357.)

This inverse relationship between solubility and temperature suggests that the use of elevated temperature during buffer exchange (e.g., UF/DF) with a calcium acetate containing formulation may result in aberrant precipitation or other potentially negative side effects. In other words, increasing the temperature for calcium acetate containing formulations would appear to reduce the buffering salt solubility. While the target concentrations of calcium acetate (Ca(Ac)₂) are relatively low in the diafiltration buffer, ˜20 mM Ca(Ac)², the local concentration of salt at an ultrafiltration membrane surface could be much higher during the UF process, especially during the overconcentration step. These high local Ca(Ac)₂ concentrations, along with the increased temperature, and the very high protein concentrations make it surprising the ultrafiltration process is able to provide a high product yield.

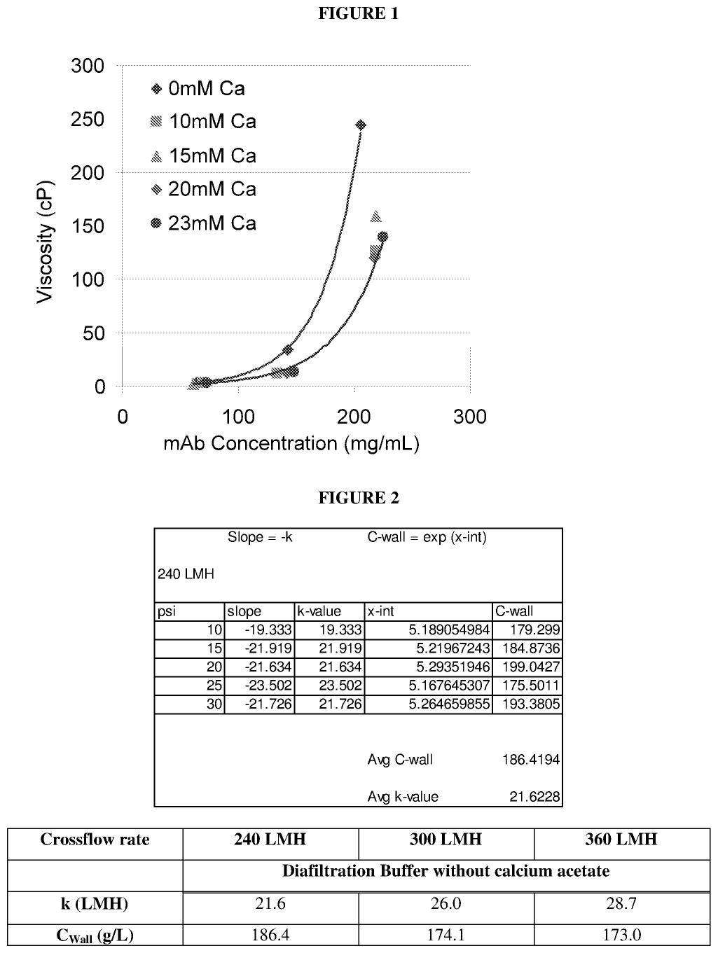

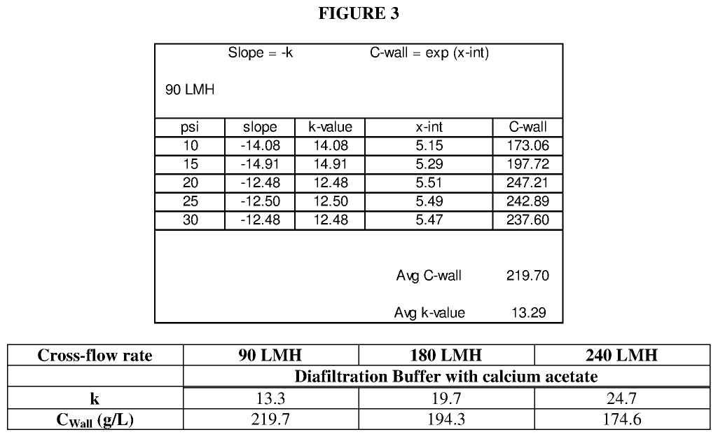

FIG. 1 shows the effect of calcium acetate on viscosity of the overconcentration material. Calcium additions between 10 mM to 23 mM reduced viscosity significantly. This is also shown in the UF/DF parameters in FIGS. 2 and 3, wherein the Cwall (max protein concentration at membrane surface) increases from 186 mg/mL to 220 mg/mL with the addition of calcium.

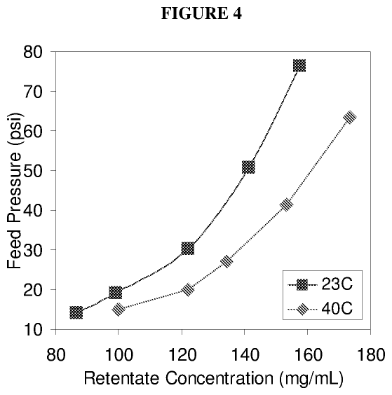

The effect of temperature on viscosity is shown in FIG. 4. Increasing temperature reduced viscosity and the resulting feed pressure for a given UF/DF condition.

Interestingly, the UF pool calcium concentration is not consistent with the diafiltration (DF) buffer after overconcentration. The diafiltration is done at 55 g/L concentration, converting the buffer to 20 mM calcium acetate, 7% sucrose, pH 5.1 over 10 diavolumes (DVs). Overconcentration increases the protein concentration by a factor of 3.3× (to 180 g/L). If the calcium was concentrated to the same degree, the 20 mM calcium DF buffer concentration would increase to at least 65 mM. Surprisingly, the experimental observation is the opposite, wherein the calcium concentration decreases below the DF buffer concentration upon overconcentration. As shown in Table 1 below, a target 20 mM Ca concentration results in an overconcentration calcium level of only 8.2 mM.

TABLE 1
Summary of calcium Concentration during various stages of production.

| | | | Actual Calcium | |
|---|---|---|---|---|
| | | Actual Calcium | concentration | |
| | Actual Calcium | concentration | (mM) (measured | Actual Calcium |
| Target Calcium | concentration | (mM) (measured | in DF over- | concentration |
| concentration | (mM) (measured | in Post-DF | concentrated | (mM) measured |
| (mM) | in DF buffer) | buffer) | retentate) | in Mock DS |
| 10 | 8.7 | 6.3 | 2.6 | 4.3 |
| 15 | 13.9 | 11.1 | 4.6 | 7.3 |
| 20 | 19.0 | 14.6 | 9.2 | 10.9 |
| 23 | 22.4 | 16.8 | 8.9 | 13.1 |

TABLE 2
Summary of calcium exclusion during overconcentration.

| Antibody concentration (mg/mL) | Actual Calcium concentration (mM) |
|---|---|
| 66.15 | 14.1 |
| 98.66 | 12.6 |
| 118.59 | 12.1 |
| 135.49 | 11.7 |
| 153.24 | 10.7 |
| 173.99 | 10.2 |

While volume exclusion effects can alter the retentate buffer composition, it is also possible that calcium ions are preferentially coordinating to the protein or partitioning due to changing solubility due to temperature increases and/or high local protein concentrations during filtration.

This Example describes a representative antibody purification process that uses an elevated temperature buffer exchange (e.g., via ultrafiltration and diafiltration (UF/DF)) step to concentrate and buffer exchange with a diafiltration buffer comprising a calcium salt to produce a final antibody pharmaceutical composition (e.g., of about 120 g/L). The ability to recover a high concentration protein from overconcentration using both higher temperature (e.g., 37° C.) and higher concentrations of a calcium salt (e.g., calcium acetate) is surprising in view of the solubility effects of calcium acetate at elevated temperatures.

1. A method of preparing an antibody pharmaceutical formulation having a viscosity of 10 cP or less comprising buffer exchanging a composition comprising the antibody with a diafiltration buffer comprising a calcium salt at a temperature greater than 30° C.

2. The method of claim 1, wherein the exchanging step occurs via ultrafiltration/diafiltration.

3. The method of claim 1, wherein the composition comprises the antibody at a concentration of least 90 mg/mL.

4. The method of claim 1, wherein the composition comprises the antibody at a concentration of least 120 mg/mL.

5. The method of claim 1, wherein the exchanging step occurs at a temperature between 30° C. and 40° C.

6. The method of any one of claims 1-5, wherein the exchanging step occurs at a temperature greater than 35° C.

7. The method of claim 1, wherein the exchanging step occurs at 37° C.

8. The method of claim 1, wherein the calcium salt is calcium acetate.

9. The method of claim 1, wherein the diafiltration buffer comprises least 20 mM calcium acetate.

10. The method of claim 9, wherein the diafiltration buffer comprises about 23 mM calcium acetate.

11. The method of claim 1, wherein the diafiltration buffer further comprises a polyol.

12. The method of claim 11, wherein the polyol is sucrose.

13. The method of claim 12, wherein diafiltration buffer comprises sucrose present at a concentration of about 1% to about 15%.

14. The method of claim 12, wherein the diafiltration buffer comprises sucrose at a concentration of about 7%.

15. The method of claim 1, further comprising the step of filtering the pharmaceutical formulation.

16. The method of claim 1, further comprising the step of aliquoting the pharmaceutical formulation into a drug product form.

17. The method of claim 1, wherein the antibody is romosozumab, abciximab, adalimumab, alemtuzumab, basiliximab, belimumab, bevacizumab, brentuximab vedotin, canakinumab, cetuximab, certolizumab pegol, daclizumab, denosumab, eculizumab, efalizumab, gemtuzumab, golimumab, ibritumomab tiuxetan, infliximab, ipilimumab, muromonab-CD3, natalizumab, nivolumab, ofatumumab, omalizumab, palivizumab, panitumumab, ranibizumab, rituximab, tocilizumab, tositumomab, trastuzumab or ustekinumab, vedolizumab.

18. The method of claim 1, wherein the pharmaceutical formulation after the exchanging step comprises about 50 mM acetate and about 12 mM calcium.


SEQUENCE LISTING (13 sequences)

SEQ ID NO: 1 - protein, 190 aa, Homo sapiens
  REGION 1..190: note = MISC_FEATURE - Human Sclerostin
QGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 2 - protein, 5 aa, Mus musculus
  REGION 1..5: note = MISC_FEATURE - romo HCDR1
DYNMH

SEQ ID NO: 3 - protein, 17 aa, Mus musculus
  REGION 1..17: note = MISC_FEATURE - romo HCDR2
EINPNSGGAGYNQKFKG

SEQ ID NO: 4 - protein, 14 aa, Mus musculus
  REGION 1..14: note = MISC_FEATURE - romo HCDR3
LGYDDIYDDWYFDV

SEQ ID NO: 5 - protein, 11 aa, Mus musculus
  REGION 1..11: note = MISC_FEATURE - romoLCDR1
RASQDISNYLN

SEQ ID NO: 6 - protein, 7 aa, Mus musculus
  REGION 1..7: note = MISC_FEATURE - romo LCDR2
YTSRLLS

SEQ ID NO: 7 - protein, 9 aa, Mus musculus
  REGION 1..9: note = MISC_FEATURE - romo LCD3
QQGDTLPYT

SEQ ID NO: 8 - protein, 107 aa, Synthetic construct [sequence omitted]

SEQ ID NO: 9 - protein, 123 aa, Synthetic construct [sequence omitted]

SEQ ID NO: 10 - protein, 236 aa, Synthetic construct
MDMRVPAQLLGLLLLWLRGARCDIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 468 aa, Synthetic construct
MDWTWRILFLVAAATGAHSEVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 214 aa, Synthetic construct
DIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 449 aa, Synthetic construct
EVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK